SEQUENCE LISTING (10 sequences)

SEQ ID NO: 1 - protein, 237 aa, Fusarium equiseti
MKFSLIAGLFAPLAMAQSLCDQYSYYANGGYEFNNNRWGQSSGSGTGCTYIDWSNSNGAGFHVDWNWSGGQDNVKAYPNAGLQIANKPLVSQVTNMQSAAAWSYKGNNVRANVAYDLFTASNPNHSTSSGDYELMIWLGRLGGVQPIGSKVGNANVEGRTWEFWSGMNGNMRVYSFVAPSPVYDFNSDVKQFWNYLANTQGYPASRQYLLTFQFGTEPFTGSGAQFKVTNFNAHVRR

SEQ ID NO: 2 - protein, 239 aa, Aspergillus niger
MKLPVSLAMLAATAMGQTMCSQYDSASSPPYSVNQNLWGEYQGTGSQCVYVDKLSSSGASWHTEWTWSGGEGTVKSYSNSGVTFNKKLVSDVSSIPTSVEWKQDNTNVNADVAYDLFTAANVDHATSSGDYELMIWLARYGNIQPIGKQIATATVGGKSWEVWYGSTTQAGAEQRTYSFVSESPINSYSGDINAFFSYLTQNQGFPASSQYLINLQFGTEAFTGGPATFTVDNWTASVN

SEQ ID NO: 3 - protein, 223 aa, Aspergillus niger
QTMCSQYDSASSPPYSVNQNLWGEYQGTGSQCVYVDKLSSSGASWHTEWTWSGGEGTVKSYSNSGVTFNKKLVSDVSSIPTSVEWKQDNTNVNADVAYDLFTAANVDHATSSGDYELMIWLARYGNIQPIGKQIATATVGGKSWEVWYGSTTQAGAEQRTYSFVSESPINSYSGDINAFFSYLTQNQGFPASSQYLINLQFGTEAFTGGPATFTVDNWTASVN

SEQ ID NO: 4 - protein, 566 aa, Unknown
  Prokaryotic organism
MKSIALGVSILMILAVVSTFSVSAQETTVLEFPGTQGSTALIDMNGDGISDFIMEINPWNIQDAQGKQIMEYDPNNNEIRFSSNLTDIVPKNSDRWIYGYPEVYFGSKPWNSKVAGGLLKLPKKVSDLSGFTLKFEYSLEHDPNLPINLAMETWLTTDQLATSGVKAGDIEIMVWLYSSKLNPAGRKIDTVTVPMIVNGRLVNESFEVWKMEGMGSGWTYFAFRLTTPLKSAEIGIDPTLFIKKVEGYIQTNVENLYMQDWEIGTEFGNPTTTSALFNWTIRNLEVNEEALLPSSTSSPEQNQTSPSNTTNLIKPGSLDVKVNSWGSATQYSCTLYLGAQYDWSVKVELKDGSEITSYWSADATEENGTVVFTPKSWNRGPTASFGFIASGDVPVESITLIVDGKVWDVWPNEAQANTTSSNQTSTQNATATNQTTNTTSTNTTSNSNQTSTDQTNTTNTTSDTLKPGSLSVSINDWGTGGQFNINLDLGGQYEWVVKVKVESPTTIGNYWSAQKSEENGWIIFTPTNWNKGPTASFGFIVNGPVTGVKQIILEVNGELWDIWTPQ

SEQ ID NO: 5 - protein, 542 aa, Unknown
  Prokaryotic organism
QETTVLEFPGTQGSTALIDMNGDGISDFIMEINPWNIQDAQGKQIMEYDPNNNEIRFSSNLTDIVPKNSDRWIYGYPEVYFGSKPWNSKVAGGLLKLPKKVSDLSGFTLKFEYSLEHDPNLPINLAMETWLTTDQLATSGVKAGDIEIMVWLYSSKLNPAGRKIDTVTVPMIVNGRLVNESFEVWKMEGMGSGWTYFAFRLTTPLKSAEIGIDPTLFIKKVEGYIQTNVENLYMQDWEIGTEFGNPTTTSALFNWTIRNLEVNEEALLPSSTSSPEQNQTSPSNTTNLIKPGSLDVKVNSWGSATQYSCTLYLGAQYDWSVKVELKDGSEITSYWSADATEENGTVVFTPKSWNRGPTASFGFIASGDVPVESITLIVDGKVWDVWPNEAQANTTSSNQTSTQNATATNQTTNTTSTNTTSNSNQTSTDQTNTTNTTSDTLKPGSLSVSINDWGTGGQFNINLDLGGQYEWVVKVKVESPTTIGNYWSAQKSEENGWIIFTPTNWNKGPTASFGFIVNGPVTGVKQIILEVNGELWDIWTPQ

SEQ ID NO: 6 - protein, 274 aa, Thermotoga maritima
MRWAVLLMVVFSALLFSSEVVLTSVGATDISFNGFPVTMELNFWNVKSYEGETWLKFDGEKVEFYADLYNIVLQNPDSWVHGYPEIYYGYKPWAGHNSGVEFLPVKVKDLPDFYVTLDYSIWYENNLPINLAMETWITRSPDQTSVSSGDAEIMVWFYNNVLMPGGQKVDEFTTTVEINGVKQETKWDVYFAPWGWDYLAFRLTTPMKEGKVKINVKDFVQKAAEVVKKHSTRIDNFEELYFCVWEIGTEFGDPNTTAAKFGWTFRDFSVEVVK

SEQ ID NO: 7 - protein, 251 aa, Thermotoga maritima
SVGATDISFNGFPVTMELNFWNVKSYEGETWLKFDGEKVEFYADLYNIVLQNPDSWVHGYPEIYYGYKPWAGHNSGVEFLPVKVKDLPDFYVTLDYSIWYENNLPINLAMETWITRSPDQTSVSSGDAEIMVWFYNNVLMPGGQKVDEFTTTVEINGVKQETKWDVYFAPWGWDYLAFRLTTPMKEGKVKINVKDFVQKAAEVVKKHSTRIDNFEELYFCVWEIGTEFGDPNTTAAKFGWTFRDFSVEVVK

SEQ ID NO: 8 - protein, 234 aa, Trichoderma reesei QM6a
MKFLQVLPALIPAALAQTSCDQWATFTGNGYTVSNNLWGASAGSGFGCVTAVSLSGGASWHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN

SEQ ID NO: 9 - protein, 218 aa, Trichoderma reesei QM6a
QTSCDQWATFTGNGYTVSNNLWGASAGSGFGCVTAVSLSGGASWHADWQWSGGQNNVKSYQNSQIAIPQKRTVNSISSMPTTASWSYSGSNIRANVAYDLFTAANPNHVTYSGDYELMIWLGKYGDIGPIGSSQGTVNVGGQSWTLYYGYNGAMQVYSFVAQTNTTNYSGDVKNFFNYLRDNKGYNAAGQYVLSYQFGTEPFTGSGTLNVASWTASIN

SEQ ID NO: 10 - protein, 221 aa, Fusarium equiseti
QSLCDQYSYYANGGYEFNNNRWGQSSGSGTGCTYIDWSNSNGAGFHVDWNWSGGQDNVKAYPNAGLQIANKPLVSQVTNMQSAAAWSYKGNNVRANVAYDLFTASNPNHSTSSGDYELMIWLGRLGGVQPIGSKVGNANVEGRTWEFWSGMNGNMRVYSFVAPSPVYDFNSDVKQFWNYLANTQGYPASRQYLLTFQFGTEPFTGSGAQFKVTNFNAHVRR

(19) United States
(12) Patent Application Publication
(10) Pub. No.: US 2022/0354132 A1
(43) Pub. Date: Nov. 10, 2022

(54) USE OF GH12 CELLULASES IN PREPARING BAKERY PRODUCTS COMPRISING RYE-FLOUR

(71) Applicant: AB Enzymes GmbH, Darmstadt (DE)

(72) Inventors: Felix FISCHER, Darmstadt (DE); Thomas HAARMANN, Darmstadt (DE); Bernard VAN GENUGTEN, Darmstadt (DE)

(21) Appl. No.: 17/637,973

(22) PCT Filed: Aug. 27, 2020
(86) PCT No.: PCT/EP2020/073996

§ 371 Date: Feb. 24, 2022

(30) Foreign Application Priority Data
Aug. 30, 2019 (EP) 19194695.3

(51) Int. Cl. A21D 8/04

(52) U.S. Cl. CPC A21D 8/042; C12Y 302/01004

(57) ABSTRACT

The invention relates to the use of a composition comprising an effective amount of at least one polypeptide having GH12 cellulase activity optionally together with conventionally used baking additives for improving at least one property of a dough comprising rye flour and/or of a bakery product prepared from said dough as well as to a method of preparing a bakery product based on rye having at least one improved property, said method comprising (i) providing a dough that has been added an effective amount of at least one polypeptide having GH12 cellulase activity or a composition comprising at least one polypeptide having GH12 cellulase activity optionally together with conventionally used baking additives, and rye flour, (ii) baking the dough for a time and temperature sufficient to yield the bakery product as well as to a baking enzyme composition for preparing rye-based bakery products comprising a polypeptide having a GH12 cellulase activity optionally together with one or more dough(s) or bakery product additives.

### RELATED APPLICATIONS

This application is the U.S. national phase entry of Int. App. No. PCT/EP2020/073996 filed Aug. 27, 2020 which published in English as WO 2021/037994 on Mar. 4, 2021 which claims priority to EP App. No. 19194695.3 filed Aug. 30, 3019 which are each incorporated by reference in its entirety.

### REFERENCE TO SEQUENCE LISTING

The present application is being filed along with a Sequence Listing in electronic format. The Sequence Listing is provided as a file entitled KRWE004004APC, created Jul. 8, 2022, which is approximately 13 kilobytes in size. The information in the electronic format of the Sequence Listing is incorporated herein by reference in its entirety.

### FIELD OF THE INVENTION

The present invention relates to the use of GH12 cellulases for improving doughs and/or bakery products based on rye as well as to a method of preparing a dough and/or a bakery product based on rye by the use of a family GH12 cellulase. Moreover, the invention relates to a rye bread-improving or rye dough-improving composition comprising a family GH12 cellulase together with conventionally used baking additives.

### BACKGROUND OF THE INVENTION

Cellulase enzymes are amongst the most widely used enzymes in industry. Generally they are applied in textile industry, detergent industry, pulp and paper industry, feed and food industry, including baking.

The known enzymes responsible for the hydrolysis of the cellulose backbone are classified into enzyme families based on sequence similarity (www.cazy.org). The enzymes with cellulase activity have been described in a number of Glycoside Hydrolase families, the most well-known of which are (GH) 5, 7, 12 and 45. Enzymes within a family share some characteristics such as 3D structure and usually share the same reaction mechanism, but they may vary in substrate specificity.

Cellulase enzymes are well known to improve dough properties in certain bread recipes, mainly in wheat breadmaking. It is well known in the art that some particular cellulase enzymes can contribute to maintain a dry dough surface to improve handling of the dough.

Cellulases comprise a catalytic domain/core (CD) expressing cellulase activity. In addition to the catalytic domain, the cellulase molecule may comprise one or more cellulose binding domains (CBDs), also named as carbohydrate binding domains/modules (CBD/CBM), which can be located either at the N- or C-terminus of the catalytic domain. CBDs have carbohydrate-binding activity and they facilitate the enzymatic action on solid substrates. The catalytic core and the CBD are typically connected via a flexible and highly glycosylated linker region (see for example WO 2010/076387).

In Luonteri, E.; Harkonen, H.; Suortti, T.; Stenholm, K.; Poutanen, K.; Tenkanen, M.; edited by: Angelino, S. A. G. F; from European Symposium on Enzymes and Grain Processing, Proceedings, 1st, Noordwijkerhout, Neth., Dec. 2-4, 1996 (1997), 142-151, Language: English, Database: CAPLUS, “Trichoderma and Aspergillus as sources of cell wall-degrading enzymes for cereal processing”, the effects of T. reesei xylanase and endoglucanase and A. terreus α-arabinosidase treatments on water-absorption capacity, viscosity, and extractability of arabinoxylans and β-glucans of rye and wheat were studied. It was found that said cell wall-degrading enzymes had positive effects both in rye and in wheat bread baking. The rye dough consistency softened both after xylanase and endoglucanase treatment. The enzymes also increased the final dough volume after fermentation and shortened the proofing time dramatically. In baked rye breads the most marked difference after enzymatic treatment was the more porous crump structure.

In Autio, K.; Parkkonen, T.; Juokslahti, T., from European Symposium on Enzymes and Grain Processing, Proceedings, 1st, Noordwijkerhout, Neth., Dec. 2-4, 1996 (1997), 152-159, Language: English, Database: CAPLUS; “Effects of cell wall-degrading enzymes on the microstructure of cereals in baking and feeding applications”, endo-β-xylanase, endo-β-glucanase, and α-arabinosidase were used to study rye cell wall structure. It was found that xylanase supplementation of rye doughs makes the dough softer, less rigid, and more adhesive compared to the control dough.

In Autio, K.; Haerkoenen, H.; Parkkonen, T.; Poutanen, K.; Siika-aho, M.; Aaman, P., from Food Science & Technology (London) (1996), 29(1 & 2), 18-27, language: English, Database: CAPLUS, DOI:10.1006/fstl.1996.0003; “Effects of purified endo-β-xylanase and endo-β-glucanase on the structural and baking characteristics of rye doughs”, the importance of arabinoxylans and β-glucans in rye baking was studied by depolymerizing these cell wall polysaccharides in situ with purified xylanase and β-glucanase. It was found that both xylanase and β-glucanase had a positive effect on the dough volume, but a negative effect on the oven rise. Addition of increasing levels of β-glucanase during baking degraded β-glucans extensively in the bread but did not influence the content or solubility of arabinoxylans.

In Weipert, D., from Getreide, Mehl und Brot (1972), 26(10), 275-80, language: German, Database: CAPLUS; “Rheologie von Roggenteigen”, the effect of 3 amylases, 3 proteases, 1 cellulase, 2 pentosanases, 1 pectinase, and 1 malt flour on rye flour were studied. It was found that the enzymes varied in their effect but all made the dough softer and gave a favourable increase in bread volume on baking.

The Master thesis by Thomas Sturm, Technische Universität München, Wissenschaftszentrum Weihenstephan für Ernshrung, Landnutzung und Umwelt, Lehrstuhl für Brau-und Getränketechnologie, submitted on Dec. 1, 2016, “Improvement of enzymatic rye flour treatment in baking”, discloses studies on the effect of the components of the product Veron Rye® which is a product commercialized by AB Enzymes GmbH, targeting at baking goods containing rye. The product is a mixture of various cellulases. An enzyme fraction A was tested which consists of endocellulase and β-mannosidase. It was found that the addition of Veron Rye® or Enzyme fraction A leads to a significantly increased dough volume during the fermentation. A higher width-to-height ratio was found in a corresponding baked bread. These effects were attributed to the endoxylanase functionalities of said enzyme fraction A. Moreover, an increase in loaf volume and a rise in the specific volume of rye/wheat breads was found. Said effects, however, were not clearly attributable to the enzymatic action of enzyme fraction A since the influence of proteins on the swelling effect and the baking effect could not be excluded. It is, moreover, emphasized in said thesis that the swelling effect and the positive baking effects seem to have two different and independent effects.

WO 2016/026850 discloses the use of a GH5 xylanase and a xylanase selected from the group consisting of GH8, GH10 and GH11 in a dough-based product. Said dough may comprise flour derived from any cereal grain including wheat, barley, rye, oat, corn, sorghum, rice and millet and, in particular, wheat and/or corn.

WO 2004/018662 corresponding to EP 03792367.9 discloses new (hemi-) cellulases and their use in baking. No specific effect of said cellulases on rye-based bakery products is disclosed.

EP 3 485 734 discloses the use of GH10 enzymes in the production of food products comprising rye whereby said rye comprises arabinoxylan.

The preparation of rye/wheat mix bread is determined chiefly by the properties of the rye flour, requiring a specialized process. Rye/wheat flour dough can be characterised by reduced elasticity, higher stickiness, lower firmness and lower form stability. Each of these properties are linearly proportional to the amount of rye flour used in the rye/wheat flour mixture. It can be said that the wheat/rye mix dough is easier to handle the higher the proportion of wheat flour is (see Weipert, 1972, cited above).

The specific properties of dough containing rye flour can be linked to the lower gluten content of rye flour, in industrial settings the properties of the dough are typically improved by adding purified gluten as an additive.

It is generally known in the art that enzymatic methods to improve doughs in rye/wheat flour are very limited, especially since enzymes that generally improve a specific deficit of wheat flour do not show the same effect in preparation of doughs from rye flour. It is undisputed that enzymes well known for improving wheat bread properties such as bread volume, dough stability, crumb texture and dough handling cannot be applied in rye-containing doughs to equal effect or even at all depending on the specific type of enzyme.

Although the composition and biochemical properties of rye flour are well characterised, there is a relatively small amount of information regarding the effect of specific types of enzymes on the constituents of rye flour and the modifications in the rye flour macromolecules necessary to obtain improved dough handling properties.

Hence, there is a need in the art for baking aids to improve the properties of rye-based doughs and rye-based bakery products. Hence, it is an object of the present invention to provide a baking aid being suitable to improve the properties of a dough based on rye flour and/or the baked product prepared therefrom. The baking aid is to improve any relevant property of a dough and/or a product obtained from said dough, particularly of a baked product, which is to be improved by the action of the baking aid according to the invention.

### SUMMARY OF THE INVENTION

The invention provides the use of a composition comprising an effective amount of at least one polypeptide having GH12 cellulase activity optionally together with conventionally used baking additives for improving at least one property of a dough comprising rye flour and/or of a bakery product prepared from said dough.

In a second aspect, the invention provides a method of preparing a bakery product based on rye having at least one improved property, said method comprising (i) providing a dough that has been added an effective amount of at least one polypeptide having GH12 cellulase activity or a composition comprising at least one polypeptide having GH12 cellulase activity optionally together with conventionally used baking additives, and rye flour, (ii) baking the dough for a time and temperature sufficient to yield the bakery product.

Still further, the invention provides a baking enzyme composition for preparing rye-based bakery products comprising a polypeptide having a GH12 cellulase activity optionally together with one or more dough or bakery product additives.

### BRIEF DESCRIPTION OF THE FIGURES

FIG. 1 shows the results of the comparative baking trial of Example 6. Bread 1 has been baked from a dough having added 80 ppm market rye enzyme. Bread 2 has been baked from a dough having added 0.6 mg EP/kg flour GH12 cellulase Type 2. Bread 3 has been baked from a dough having added 1 mg EP/kg flour GH10 cellulase Type 1. The photographs of the corresponding cross-sections of the respective breads show that the bread according to the invention (bread 2) is rounder and has a fine pore structure.

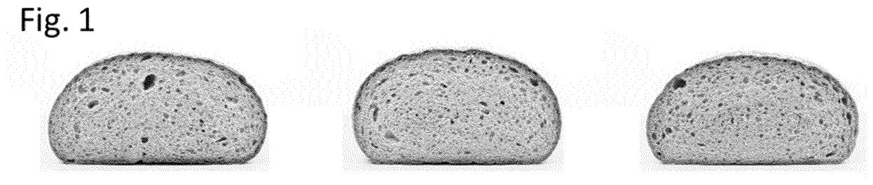

FIG. 2 shows the results of the swelling test according to Example 8. The test results are presented in the order in which the experiments appear in Table 7.

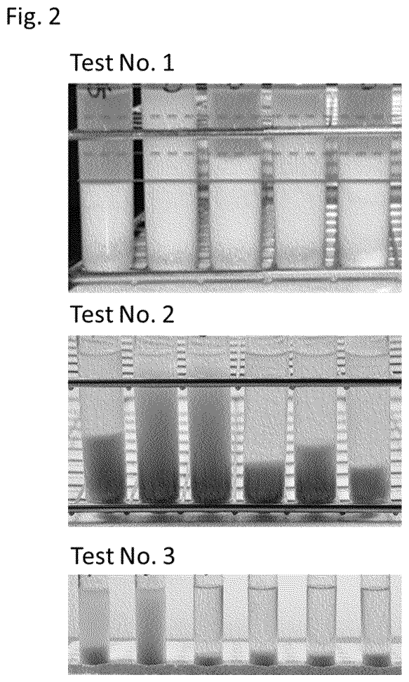

FIG. 3 shows the results of the comparative baking trial of Example 9. Bread 1 has been baked from a dough having no added enzyme. Bread 2 has been baked from a dough having added 4.4 mg EP/kg flour GH12 cellulase Type 2. The photographs of the corresponding cross-sections of the respective breads show that the bread according to the invention (bread 2) is rounder and has more volume.

FIG. 4 shows the content of https://www.enzyme-database.org/query.php?ec=3.2.1.4

### DETAILED DISCLOSURE OF THE INVENTION

The present invention generally relates to the use of a GH12 cellulase for improving at least one property of a dough comprising rye flour and/or of a bakery product prepared from said dough. As outlined above, the prior art mentions various approaches to improve the properties of doughs and/or bakery products by use of various enzymes. However, none of the prior art references discloses the use of GH12-family enzymes for improving the properties of doughs and/or bakery products, let alone for improving the properties of rye-based doughs and/or rye-based bakery products. Comprehensive studies characterising the functionality of GH12 cellulases on well characterised and pure substrates have been done (Sandgren, M., Stahlberg, J., & Mitchinson, C. (2005)), Structural and biochemical studies of GH family 12 cellulases: improved thermal stability, and ligand complexes. Progress in biophysics and molecular biology, 89(3), 246-291). These studies show that GH12 cellulases have specific requirements with respect to the composition of the substrate, different from other common cellulase families such as GH5, GH7 or GH45 cellulases (E. Vlasenko, M. Schulein, J. Cherry, F. Xu (2010). Substrate specificity of family 5, 6, 7, 9, 12, and 45 endoglucanases. Bioresource Technology, 101, 2405-2411). The reason for these differences in specificity is thought to be due to the three dimensional structure of the enzyme, which is directly related to the amino acid sequence. Up until now, the relation of GH12 enzymes to the substrate in different grains used for breadmaking has not been well documented.

It was therefore surprising that a polypeptide having GH12 cellulase activity can advantageously be used to improve the properties of a rye-based dough and/or bakery products obtained from said dough. An improved property of a rye-based dough and/or rye-based bakery product denotes any property of a dough and/or a product obtained from the dough, particularly a baked product, which is improved by the action of a polypeptide having GH12 cellulase activity according to the invention relative to a dough or product in which the polypeptide having GH12 cellulase activity is not incorporated. The improved property may include, but is not limited to, increased strength of the dough, increased elasticity of the dough, increased stability of the dough, reduced stickiness of the dough and improved extensibility of the dough, improved flavour of the baked product, improved anti-staling of the baked product, improved height-to-width ratio of the final bread, improved crumb structure, improved elasticity and improved softness of the dough. The improved property may be determined by comparison of a dough and/or a baked product prepared with and without addition of a polypeptide having GH12 cellulase activity according to the invention in accordance with methods which are described below or are known in the art. Organoleptic qualities may be evaluated using procedures well established in the baking industry and may include, for example, the use of a panel of trained taste testers.

The term “increased strength of the dough” is defined herein as the property of a dough that generally has more elastic properties and/or requires more work input to mould and shape. The term “increased elasticity of the dough” as defined herein is the property of a dough, which has a higher tendency to regain its original shape after being subjected to a certain physical strain. The term “increased stability of the dough” is defined herein as the property of the dough that is less susceptible to mechanical abuse, thus better maintaining its shape and volume.

The term “reduced stickiness of the dough” is to denote the property of a dough that has less tendency to adhere to surfaces, e.g. in the dough-production machinery, and is either evaluated by the skilled test baker or measured by the use of a texture analyser as known in the art. The term “improved extensibility of a dough” is to denote a dough that can be subjected to an increased strain or stretching without rupture. The term “improved machinability of the dough” is defined herein as the property of a dough that is generally less sticky and/or more firm and/or more elastic. The term “increased volume of the baked product” is measured as the specific volume of a given loaf of a bread (volume/weight) determined typically by the traditional rapeseed displacement method. The term “improved crumb structure of the baked product” is defined herein as the property of the baked product with finer and/or thinner cell walls in the crumb and/or more uniform/homogenous distribution of cells in the crumb and is usually evaluated empirically by the skilled tester.

The term “improved softness of the baked product” is the opposite of firmness and is defined herein as the property of a baked product that is more easily compressed and is evaluated empirically by the skilled test baker or measured by the use of a texture analyser as known in the art. The term “improved flavour of the baked product” is evaluated by a trained test panel. The term “improved anti-staling of the baked product” is defined herein as the properties of the baked product that have a reduced rate of deterioration of quality parameters, e.g. softness and/or elasticity during storage.

According to the invention, a polypeptide having GH12 cellulase activity is used for improving at least one property of a rye-based dough and/or a baked product therefrom. Preferably, the use of a polypeptide having GH12 cellulase activity improves at least one property both of a rye-based dough and of a baked product therefrom. Preferably said property is selected from the stability, the stickiness and the softness of the dough and/or the shape, the crumb structure and the height/width ratio of the bakery product.

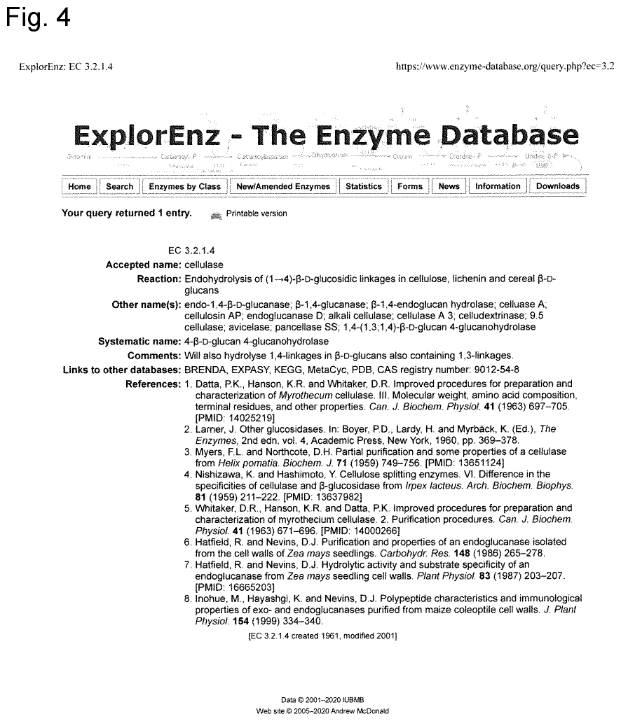

A polypeptide having GH12 cellulase activity is to denote a polypeptide belonging to the glycoside hydrolases of family 12. They (the glycoside hydrolases of family 12) include endo-β-1,4-glucanase (EC 3.2.1.4), xyloglucan endohydrolase (EC 3.2.1.151) and endo-β-1,3-1,4-glucanase (EC 3.2.1.73) (see: www.cas zypedia.org/index.php/Glycoside_Hydrolase_Family_12). Generally a cellulase is an enzyme protein that catalyses the endo-hydrolases of (1->4)-beta-D-glucosidic linkages in cellulose, lichenin and cereal beta-D-glucans (EC 3.2.1.4) (see: https://www.enzyme-database.org/query.php?ec=3.2.1.4 (enclosed as FIG. 4)).

Corresponding enzymes are known in the art. A corresponding enzyme is, for example, described in WO 2004/018662 (corresponding to EP 03 792 367.9). Said document mentions the use of said enzyme in improving properties of a dough and/or a baked product but does not specifically mention the improvement of rye-based doughs or baked products. The polypeptide having GH12 cellulase activity may be derived from any organism, for example selected from the group consisting of Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Streptomyces, Talaromyces, Thermoascus, Thielavia, Tolypocladium, Trametes, Trichoderma, Hyprocrea jecorina, Trichoderma reesei, Trichoderma viride, Trichoderma harzianum, Aspergillus niger, Aspergillus oryzae, Humicola insolens, Humicola grisea, Streptomyces sp., Streptomyces violaceoruber, Bacillus licheniformis, Bacillus amyloliquefaciens, Bacillus pumilus, Bacillus sp, Thermotoga maritima, Xanthomonas, Clostridium, Actinoplanes, Proteus, Erwinia, Shigella, Haemophilus, Streptococcus, Salmonella, Pseudomonas, Rhizobium, Agrobacterium, or Vibrio. The organisms may contain or express one or more genes coding for polypeptides having GH12 cellulase activity. Said multiple forms are usually denoted as type 1, type 2 etc. enzymes.

Polypeptides having GH12 cellulase activity are well-known in the art and said polypeptides may be used as such or variants of said polypeptides may be used which have, for example, single or multiple amino acid substitutions, deletions and/or insertions, provided that they have the GH12 cellulase activity. Moreover, hybrid polypeptides of said GH12 cellulases may be used in which a region of a GH12 cellulase is used at the N terminus or the C terminus of a region of another polypeptide, provided that the GH12 cellulase activity is still maintained. There are various subtypes of the GH12 cellulase peptides which may also be used and which are referred to in the following. The following polypeptides having GH12 cellulase activity are preferably used according to the present invention. These polypeptides belong to the glycosides hydrolases of family 12 as defined above, have GH12 cellulase activity but vary in their sequences. These polypeptides are referred to as GH12 type 1 to 5 or alternatively as GH12 molecules 1 to 5.

These are the following polypeptides having GH12 cellulase activity selected from the following cellulases having or comprising the respective sequence: GH12 Type 1 (SEQ ID NO:1/with signal peptide or SEQ ID NO:10 without signal peptide), GH12 Type 2 (SEQ ID NO:2/with signal peptide or SEQ ID NO:3/without signal peptide, UniProt Ref. 074705), GH12 Type 3 (SEQ ID NO:4/with signal peptide or SEQ ID NO:5/without signal peptide, GenBank Ref. CRL09147), GH12 Type 4 (SEQ ID NO:6/with signal peptide or SEQ ID NO:7/without signal peptide, NCBI Reference sequence WP_004081891), GH12 Type 5 (SEQ ID NO:8/with signal peptide or SEQ ID NO:9/without signal peptide, NCBI Reference sequence XP_006967891) or a polypeptide having GH12 cellulase activity and having at least 60%, preferably at least 70%, more preferably at least 80%, more preferably at least 90%, still more preferably at least 95%, still more preferably at least 98% sequence identity to any of SEQ ID NOS:1 to 10. The above preferred polypeptides having GH12 cellulase activity may either be used singly or in a mixture of at least two of said cellulases. Functional equivalents of said sequences may also be used such as the above sequence having mutations, substitutions, deletions and/or insertions, provided they have the claimed GH12 cellulase activity. Moreover, the above preferred polypeptides may be contained in a larger molecule such as a fusion protein, provided that they still have the claimed activity, i.e. polypeptides having GH12 cellulase activity and comprising any of SEQ ID NOS:1 to 10 may also be used according to the invention. Further functional equivalents of said proteins can also be identified by using the above sequences to screen combinatorial libraries.

The most preferred polypeptide having GH12 cellulase activity for the use of the invention is the GH12 type 2 molecule.

The degree of sequence identity is preferably determined in such a way that the number of residues of the shorter sequence which is involved in the comparison and has a “corresponding” counterpart in the other sequence is determined. For the purposes of the present invention the identity is preferably determined in the usual manner by using the usual algorithms. Similar, preferably identical sequence counterparts can be determined according to the invention as homologue sequences by means of known computer programs. An example of such a program is the program Clone Manager Suite, which includes the program part Align Plus and is distributed by Scientific & Educational Software, Durham, N.C., U.S.A. A comparison of two DNA sequences or amino acid sequences as defined above is thereby carried out under the option local alignment either according to the FastScan—MaxScore method or according to the Needleman-Wunsch method, keeping the default values. The program version “Clone Manager 7 Align Plus 5” with the functions “Compare Two Sequences/Local Fast Scan-Max Score/Compare DNA sequences” or for amino acids “Compare Two Sequences/Global/Compare sequences as Amino Acids” was particularly used to calculate the identity according to the invention. The algorithms made available from the following sources were thereby used: Hirschberg, D. S. 1975. A linear space algorithm for computing maximal common subsequences. Commun Assoc Comput Mach 18:341-343; Myers, E. W. and W. Miller. 1988. Optimal alignments in linear space. CABIOS 4:1, 11-17; Chao, K-M, W. R. Pearson and W. Miller. 1992. Aligning two sequences within a specified diagonal band. CABIOS 8:5, 481-487.

The production of such variants is generally known in the state of the art. For example, amino acid sequence variants of the polypeptides may be produced by mutation in the DNA. Processes for mutagenesis and changes in the nucleotide sequence are well known in the state of the art (cf. for example Tomic et al. NAR, 18:1656 (1990), Giebel and Sprtiz NAR, 18:4947 (1990)).

Details on appropriate amino acid substitutions which do not negatively influence the biological activity of the protein of interest can be found in the model by Dayhoff et al., Atlas of Protein Sequence and Structure, Natl. Biomed. Res. Found., Washington, D.C. (1978). Conservative substitutions such as the substitution of an amino acid by another one with similar properties are preferred. These substitutions may be divided into two main groups with altogether four subgroups, and a substitution in each subgroup is referred to as conservative substitution, which does preferably not influence the activity or the folding of the protein.

| | | |
|---|---|---|
| aliphatic | non-polar | G A P |
| | | I L V |
| | polar and uncharged | C S T M N Q |
| | polar and charged | D E |
| | | K R |
| aromatic | | H F W Y |

The polypeptide having GH12 cellulase activity is added to any mixture of dough ingredients, to the dough or to any of the ingredients to be included in the dough, i.e. it may be added in any step of the dough preparation and may be added in one, two or more steps, where appropriate. Usually, the polypeptide having GH12 cellulase activity is added in an amount corresponding to 0.001 to 500 mg enzyme protein per kilogram of flour, preferably 0.01 to 100 mg enzyme protein per kilogram of flour, preferably 0.05-50 mg enzyme protein per kilogram of flour, more preferably 0.05-20 mg enzyme protein per kilogram of flour, 2.5 to 15 mg enzyme protein per kilogram of flour, 0.1-10 mg enzyme protein per kilogram of flour, still more preferably 0.1-5 mg enzyme protein per kilogram of flour, even still more preferably 0.5-2.5 mg protein per kilogram of flour.

A particular dosage may be applied if selectively either the properties stickiness and softness of the dough or the baking properties volume and height/width ratio of the bakery product are to be improved. For the improvement of the properties of the dough (stickiness and softness) a preferred dosage of the GH12 cellulase is 0.001 to 2.5 mg enzyme protein per kilogram of flour, preferably 0.001 to 1 mg enzyme protein per kilogram of flour, more preferably 0.001 to 0.5 mg enzyme protein per kilogram of flour, and still more preferably 0.01 to 0.5 mg enzyme protein per kilogram of flour. If the properties volume and height/width ratio of the bakery product are to improve a preferred dosage of GH12 cellulase is 1 to 500 mg enzyme protein per kilogram of flour, preferably 1 to 100 mg enzyme protein per kilogram of flour, more preferably 1 to 50 mg enzyme protein per kilogram of flour, still more preferably 5 to 50 mg enzyme protein per kilogram of flour.

The most suitable dosage of the polypeptide having GH12 activity can easily be determined by a person skilled in the art on the basis of the present disclosure and in relation to the specific bread recipe. As evident from the examples, there may be slight differences in the preferred dosage depending on the type of a polypeptide having GH12 cellulase activity as used. A preferred dosage range for the above or any other Type 1 GH12 cellulase is 2.5 to 15 mg enzyme protein per kilogram of flour and a preferred dosage range of the above or any other Type 2 cellulase enzyme may vary between 0.05 and 20 mg enzyme protein per kilogram flour. The most suitable dosage of the polypeptide having GH12 cellulase activity is of course subject to the type/origin/batch etc. of the respective flour, but may easily be determined by a person skilled in the art.

A more preferred dosage range for the above or any other Type 1 GH12 cellulase is 2.5 to 7.5 mg enzyme at 50% to 100% percentage of rye flour used in the recipe. A furthermore preferred dosage range for the above or any other Type 1 GH12 cellulase is 7.5 to 15 mg enzyme at 10% to 50% percentage of rye flour used in the recipe. A more preferred dosage range for the above or any other Type 2 GH12 cellulase is 0.05 to 1.5 mg enzyme at 50% to 100% percentage of rye flour used in the recipe. A more preferred dosage range for the above or any other Type 1 GH12 cellulase is 1.5 to 20 mg enzyme at 10% to 50% percentage of rye flour used in the recipe.

The enzyme may be added as such or as a constituent of a dough-improving or a bread-improving composition, either to flour or other dough ingredients or directly to the mixture from which the dough is to be made. Further enzymes may be added in combination with a polypeptide having GH12 cellulase activity. Examples of such enzymes are further cellulases, a glycosyl transferase, in particular, 1-α-glucano-transferase or 4-α-glucano-transferase, a hemicellulase, for example a pentosanase such as xylanase, a lipase, an oxidase, for example a glucose oxidase, a peroxidase, a protease, a peptidase, a transglutaminase, and/or an amylolytic enzyme, in particular an amylolytic enzyme with α-1,4-endo-activity, a β-amylase, an amyloglucosidase, a maltogenic amylase, a cyclodextrine glucanotransferase and the like. The other enzyme components may be of any origin including mammalian and plant and preferably of microbial (including bacterial and fungal) origin. The enzymes as well as the polypeptide having GH12 cellulase activity may be obtained by conventional techniques used in the art.

The amylase may be fungal or bacterial, e.g., a maltogenic alpha-amylase from B. stearothermophilus or an alpha-amylase from Bacillus, e.g., B. licheniformis or B. amyloliquefaciens, a beta-amylase, e.g., from plant (e.g. soy bean) or from microbial sources (e.g. Bacillus), or a fungal alpha-amylase, e.g. from A. oryzae.

Suitable commercial maltogenic alpha-amylases include NOVAMYL™ (available from Novozymes A/S) and VERON® MAC (available from AB Enzymes GmbH). Suitable commercial fungal alpha-amylase compositions include, e.g., FUNGAMYL 2500 SG™, AMYL ULTRA SG™ (available from Novozymes A/S), VERON® M4, VERON® 1000, VERON® AC, VERON® BA, VERON® (available from AB Enzymes GmbH).

The xylanase may be a fungal or bacterial xylanase, in particular an Aspergillus niger xylanase such as VERON® 191S or Bacillus subtilis xylanase such as VERON® RL (Available from AB Enzymes GmbH).

The glucose oxidase may be a fungal glucose oxidase, in particular an Aspergillus niger glucose oxidase (such as GLUZYME™, available from Novozymes A/S, Denmark), VERON® Oxibake-ST (available from AB Enzymes GmbH).

The protease may be from Bacillus, e.g., B. subtilis, such as VERON® P (available from AB Enzymes GmbH).

The phospholipase may have phospholipase A1, A2, B, C, D or lysophospholipase activity, it may or may not have lipase activity. It may be of animal origin, e.g. from pancreas, snake venom or bee venom, or it may be of microbial origin, e.g. from filamentous fungi, yeast or bacteria, such as Aspergillus or Fusarium, e.g. A. niger, A. oryzae or F. oxysporum. A preferred lipase/phospholipase from Fusarium oxysporum is disclosed in WO 98/26057. Also, the variants described in WO 00/32758 may be used.

Suitable phospholipase compositions are VERON® Hyperbake-ST (available from AB Enzymes GmbH).

Preferred enzymes to be combined with a GH12 cellulase are xylanases/endoxylanases, preferably in an amount of 0.1 to 20 mg enzyme protein/kg flour, alpha-amylases, preferably in an amount of 0.1 to 10 mg enzyme protein/kg flour, and multigenic amylases, preferably in an amount of 0.5 to 50 mg enzyme protein/kg flour.

The dough comprises rye or may consist thereof. Advantageously the dough contains 10 to 100% rye flour. The terms “rye-based dough” or “dough comprising rye” are to refer to a mixture of flour and other ingredients which contain a gist of or more than 10% rye flour, 20% rye flour, 25% rye flour, 30% rye flour, 35% rye flour, 40% rye flour, 45% rye flour, preferably 50% rye flour, more preferably 60% rye flour, more preferably 70%, still more preferably 80% rye flour, still more preferably 90% rye flour. The dough contains preferably 30 to 90% rye flour, more preferably 40 to 90% rye flour, still more preferably 50 to 90% rye flour, still more preferably 60 to 90% rye flour, still more preferably 70 to 90% rye flour, more preferably 30 to 80% rye flour, still more preferably 40 to 80% rye flour, still more preferably 50 to 80% rye flour, still more preferably 60 to 80% rye flour, and more preferably 70 to 80% rye flour. Most preferably, the dough contains 30 to 70%, most preferably 50 to 70% rye flour. A preferred embodiment is also a dough containing 100% rye flour.

The dough may be fresh, frozen or prebaked. The other flours comprised in the dough apart from the rye flour may be of any cereal grain including wheat, barley, emmer, triticale, oat, corn, sorghum, rice, spelt, and millet, in particular wheat and/or corn. The dough may also comprise other convention dough ingredients, for example proteins such as milk powder, sugar, gluten and soy, eggs (whole eggs, egg yolks or egg whites), an oxidant such as ascorbic acid, potassium bromate, potassium iodate, acyldicarbonamide or ammonium persulphate, an amino acid such as L-cystein, a sugar, a salt such as sodium chloride, calcium acetate, sodium sulphate or calcium sulphate. The dough may also comprise an emulsifier selected from the group consisting of diacetyl tartaric acid esters of monoglycerides, sodium stearyl lactylate, calcium stearyl lactylate, ethoxylated mono and diglycerides, polysorbates, succinolated monoglycerides and mixtures thereof. The dough may contain rye-based or wheat-based sourdough, gums and hydrocolloids such as guar, xanthan or cellulose. Malt (active and inactive), yeast, and water.

Depending on the desired final product, the recipe may contain further ingredients conventionally used according to the desired bread or bakery product. Further ingredients may be, for example, spices, dried fruits, nuts, chocolate, colouring agents, aromatising agents, etc.

A baked product obtained from said dough is any product prepared from a dough either of a soft or a crisp character. Examples of baked products, whether of a white, light or dark type, which may be advantageously produced according to the present invention are rye loaves, rye buns, rye rolls, whole-grain loaves, rye pretzels or rye pizza, crisp bread.

The handling of the dough and/or baking is performed in any suitable manner for the dough and/or baked product in question, typically including the steps of kneading the dough, subjecting the dough to one or more proofing treatments and baking the product under suitable conditions, i.e. at a suitable temperature and for a sufficient period of time. For example, the dough may be prepared by using a normal straight dough process, a sour dough process, an overnight dough process, a low-temperature and long-time fermentation method, a frozen dough method and the like.

Particularly preferred methods for preparing a bakery product based on rye according to the invention are mixing the flour with water, yeast and other ingredients, where the enzyme is added to the flour directly or as component in a bread improver or sourdough. Preferably, the ingredients are mixed in a kneading machine until the dough is fully developed. After mixing the temperature is being measured and the dough gets evaluated by a professional baker on its properties which are, stickiness, elasticity, extensibility and softness. The dough is being covered with plastic to prevent the surface from drying out. The dough is being left 20 minutes for bench time. After resting, the dough is being scaled, shaped and put into a breadbasket. The dough is being proofed in a controlled environment for 45-75 minutes at 32° C. and 80% relative humidity. After proofing the dough is being transferred into an oven and baked for 50-60 minutes at 260° C. with steam and a falling temperature to 220° C. After baking, the fresh loaf is being left at ambient for cooling down. Final evaluation and volume measurement is being done when the bread reaches room temperature.

Preferred dough compositions are as follows: 10-100% rye flour, 0-90% other grains, further containing water, yeast, possible other additives [as in the list above] and enzymes.

Preferred recipes are those as used in the examples of the invention in relation to the ingredients and to the added enzyme. The amount of the ingredients may vary and the respective products may also be prepared with other processes of a dough preparation and baking that are usual in the art. This means that the features of the recipe and the enzyme as used are not inextricably linked with the respective dough preparing or baking process as exemplified in the examples.

The present invention is further described by the following examples:

### EXAMPLES

### General Methods

### Methods for the Preparation and Evaluation of the Properties of the Dough and Bakery Products (Baking and Evaluation Process):

### General Procedure for Making the Breads

In the following a general outline of an exemplary procedure for making the breads is given.
1) Weighing all ingredients. Flours, salt, yeast, citric acid, gluten and other ingredients and the enzymes are added into a plastic bowl.
2) The ingredients are added to a spiral mixer Diosna SP12. The water temperature is adjusted to 34° C. and added into the mixer. All ingredients are mixed at 50 Hz for 6 minutes and 60 Hz for 1 minute.
3) The dough is taken from the mixer with help of a plastic scraper and left to rest at room temperature for 20 minutes.
   During resting, the dough temperature is measured using a standard thermometer (Testo 926 with PT100) to make sure that each dough has the same temperature (a variation between doughs of 1° C. is allowed). Dough temperature after mixing should be at 28-30° C.
   The dough is manually evaluated (see below). Each dough is divided into exactly 800 gr pieces using scales and added into a bowl for fermentation. The dough is fermented at 32° C. and 80% relative humidity for 45 minutes and 60 minutes.
4) Afterwards, the dough is baked at 260° C. for 60 minutes.
5) After the breads are cooled down, the volume is measured using a BVM 6630 (Perten Instruments). Final volume is the average of at least three breads.

### Manual Dough and Bread Evaluation

Dough properties are evaluated directly after mixing, during the 20 minutes bench time and shaping. A scale of 1 to 10 indicates the dough properties, where dough 1 (reference) is given score 5 for all parameters and all other doughs are compared to dough 1. A higher number means that the described parameter is more or higher compared to the reference. A lower stickiness and softness is preferable and wanted which is represented by a lower number. The following properties of the bread are evaluated after baking.

| Parameter | Definition | Scale |
|---|---|---|
| Stickiness | Degree to which the | Less sticky 0-4 |
| after mixing | dough sticks to the | Same as control 5 |
| | hands when dough is | More sticky 6-10 |
| | pressed by the palm of | |
| | the hand, tested directly | |
| | after removing dough | |
| | from the mixer. | |
| Stickiness | Degree to which the | |
| after resting | dough sticks to the | |
| | hands when pressed by | |
| | the palm of the hand, | |
| | tested 5 to 10 minutes | |
| | after removing the | |
| | dough from the mixer | |
| Extensibility | The ability to stretch a | Less extensible 0-4 |
| | dough by pulling a piece | Same as control 5 |
| | of dough on both ends | More extensible 6-10 |
| | without breaking | |
| Elasticity | The ability of a dough to | Less elastic 0-4 |
| | return to its original | Same as control 5 |
| | shape after being pulled | More elastic 6-10 |
| Softness | The amount of force | Less soft 0-4 |
| | needed to depress the | Same as control 5 |
| | surface of a dough | More soft 6-10 |
| | piece | |
| Bread volume | Volume of a complete | Measured in ml |
| | loaf as measured using | |
| | volume scanner (Perten | |
| | Instruments) | |
| Bread shape | The height versus width | Just Round, Somewhat |
| | of a cross-section of | round, Round = higher |
| | bread | height vs. width at |
| | | equal volume |
| | | compared to control |
| | | Normal = No change |
| | | in height vs. width |
| | | at equal volume |
| | | compared to control |
| | | Just flat, Somewhat |
| | | Flat, Flat = more width |
| | | vs. hight at equal |
| | | volume compared to |
| | | control |

For the loaf shape the properties flat, somewhat flat, just flat, normal, just round, somewhat round and round are used.

### Example 1: Dough Properties, Bread Volume and Shape Using GH12 Cellulase Type 1

A dosage range of GH12 cellulase Type 1 against a Market Rye enzyme product was tested. A bread was baked using the following recipe according to the described baking and evaluation process.

### Recipe

Rye Flour: Grade 1150, Germany
Wheat Flour: Grade 550, Germany

| Ingredient | Amount (w/w on total flour) |
|---|---|
| Water | 80% |
| Salt | 2% |
| Fresh yeast | 2% |
| Citric acid | 1.1% (on rye flour weight) |
| Gluten | 1% |
| Ascorbic acid | 80 ppm |

The enzymes doses are in ppm based on the weight of the flour. EP=enzyme protein.

80% Rye flour, 20% Wheat flour

| | 80 ppm | | | |
|---|---|---|---|---|
| | Reference | 2.5 mg EP/kg | 7.5 mg EP/kg | 15 mg EP/kg |
| | Market Rye | flour | flour | flour |
| | enzyme | GH12 Type 1 | GH12 Type 1 | GH12 Type 1 |
| Stickiness | 5 | 3 | 3 | 5 |
| after mixing | | | | |
| Stickiness | 5 | 3 | 3 | 5 |
| after resting | | | | |
| Extensibility | 5 | 5 | 5 | 5 |
| Elasticity | 5 | 5 | 5 | 5 |
| Softness | 5 | 4 | 4 | 5 |
| Bread volume | 1335 | 1290 | 1422 | 1520 |
| [ml] | | | | |
| Bread shape | Somewhat | Round | Just round | Just round |
| | flat | | | |

Conclusion: The results show that an optimal dosage of 2.5 mg EP/kg flour to 7.5 mg EP/kg flour gives decreased stickiness of the dough after mixing and after around 10 minutes resting time, compared to the reference dough. The volume is slightly lowered at 2.5 mg EP/kg flour but with better bread shape, the volume is increased again when using 7.5 mg EP/kg flour.

### Example 2: Comparison Between GH12 Cellulase and the Market Rye Enzyme Product in Baking

A bread was baked using the following recipe and process:

### Recipe

Rye Flour: Grade 1150, Germany
Wheat Flour: Grade 550, Germany

| Ingredient | Amount (w/w on total flour) |
|---|---|
| Water | 60% |
| Salt | 1.5% |
| Fresh yeast | 3% |
| Ascorbic acid | 50 ppm |
| VERON® M4 | 5 ppm |

A bread was prepared using GH12 Type 2 cellulase in comparison to a blank dough without added rye enzyme. The enzyme doses and the results are given in the following Table 1:

TABLE 1
10% Rye flour, 90% Wheat flour

| | Reference | 18 mg EP/kg flour |
|---|---|---|
| | No enzyme | GH12 Type2 |
| Stickiness after mixing | 5 | 4 |
| Stickiness after resting | 5 | 4 |
| Extensibility | 5 | 6 |
| Elasticity | 5 | 5 |
| Softness | 5 | 4 |
| Bread volume [ml] | 1911 | 2096 |
| Bread shape | Somewhat flat | Round |

The results show that an optimal dosage of 18 mg EP/kg flour gives decreased stickiness of the dough after mixing and after around 10 minutes resting time, compared to the reference dough. The resulting bread volume is significantly higher.

### Example 3: Baking Trial Using Whole-Grain Rye Flour

A bread was baked according to the described baking and evaluation process using the following recipe:

### Recipe

Rye Flour Whole Grain: Finland
Wheat Flour: Grade 550, Germany

| Ingredient | Amount (w/w on total flour) |
|---|---|
| Water | 82% |
| Salt | 2% |
| Fresh yeast | 2% |
| Citric acid | 1.1% (on rye flour weight) |
| Ascorbic acid | 100 ppm |

The following Table 2 presents the results using a GH12 Type 2 against a reference without enzyme.

TABLE 2
80% Rye flour Whole Grain, 20% Wheat flour

| | Reference | 1.4 mg EP/kg flour |
|---|---|---|
| | No enzyme | GH12 Type2 |
| Stickiness after mixing | 5 | 5 |
| Stickiness after resting | 5 | 4 |
| Extensibility | 5 | 5 |
| Elasticity | 5 | 5 |
| Softness | 5 | 5 |
| Bread volume [ml] | 1210 | 1285 |
| Bread shape | Normal | Normal |

Conclusion: The results show that at 1.4 mg EP/kg flour GH12 Type 2 dosage, an improved volume is obtained compared to the blank dough without enzyme. After resting the dough for 10 minutes, the dough was less sticky which makes the dough easier to handle and process. The result emphasizes that the positive effect of GH12 Type 2 can be shown also using Whole Grain rye flour.

### Example 4: GH12 Cellulase Type 2 in Baking

Bread was baked according to the described baking and evaluation process using the following recipe:

### Recipe

Rye Flour: Grade 1150, Germany
Wheat Flour: Grade 550, Germany

| Ingredient | Amount (w/w on total flour) |
|---|---|
| Water | 70% |
| Salt | 2% |
| Fresh yeast | 2% |
| Citric acid | 1.1% (on rye flour weight) |
| Ascorbic acid | 80 ppm |

Table 3 shows the results of a baking trial using GH12 cellulase Type 2 against a reference without enzyme.

TABLE 3
60% Rye flour, 40% Wheat flour

| | Reference | 1.4 mg EP/kg flour |
|---|---|---|
| | No enzyme | GH12 Type2 |
| Stickiness after mixing | 5 | 5 |
| Stickiness after resting | 5 | 5 |
| Extensibility | 5 | 5 |
| Elasticity | 5 | 5 |
| Softness | 5 | 5 |
| Bread volume [ml] | 1655 | 1709 |
| Bread shape | Somewhat flat | Round |

The results show that a 1.4 mg EP/kg flour dosage of GH12 cellulase type 2 lead to a higher bread volume and to a round bread shape.

Conclusion: By using GH12 Type 2 the shape and volume of the rye/wheat mix bread could be significantly improved without negatively affecting dough properties.

### Example 5: Comparative Baking Trial with GH12 Cellulase Type 1 and Type 2 Against Reference Market Enzymes

Bread was baked according to the described baking and evaluation process using the following recipe:

### Recipe

Rye Flour: Grade 1150, Germany
Wheat Flour: Grade 550, Germany

| Ingredient | Amount (w/w on total flour) |
|---|---|
| Water | 80% |
| Salt | 2% |
| Fresh yeast | 2% |
| Citric acid | 1.1% (on rye flour weight) |
| Ascorbic acid | 60 ppm |

The results are shown in the following Table 4.

TABLE 4
80% Rye flour, 20% Wheat flour

| | Reference | Reference | | |
|---|---|---|---|---|
| | 100 ppm | 400 ppm | 5 mg EP/kg | 0.4 mg EP/kg |
| | Market | Market Rye | flour | flour |
| | Xylanase | enzyme | GH12 Type 1 | GH12 Type2 |
| Stickiness | 5 | 5 | 4 | 3 |
| after mixing | | | | |
| Stickiness | 5 | 5 | 4 | 2 |
| after resting | | | | |
| Extensibility | 5 | 5 | 5 | 5 |
| Elasticity | 5 | 5 | 5 | 5 |
| Softness | 5 | 5 | 5 | 3 |

Conclusion: The above experiment shows that use of GH12 cellulase leads to significantly improved dough properties, since the doughs having an addition of a GH12 cellulase have a lower stickiness after mixing and after resting and are firmer, therefore easier to handle and process. Moreover, the addition of the enzyme does not have a negative effect on other dough properties such as extensibility, elasticity and softness.

### Example 6: Comparative Baking Trial with Cellulase GH 12 Type 2 and GH10 Type 1 Against Reference Market Rye Enzyme

A bread was baked according to the described baking and evaluation process using the recipe mentioned in table 5.
Dough No. 1=80 ppm Market Rye enzyme
Dough No. 2=0.6 mg EP/kg flour GH12 Type 2
Dough No. 3=1 mg EP/kg flour GH10 Type 1

TABLE 5

| | 70% Rye Flour T1150, 30% Wheat | | |
|---|---|---|---|
| | Flour T550, 80% Water, 2% salt,2% fresh yeast, | | |
| Recipe | 2% vital wheat gluten, 1.1% Citric acid (on rye flour) | | |
| Dough No. | 1 | 2 | 3 |
| Dough temperature | 28.1 | 28.0 | 28.5 |
| [° C.] | | | |
| Stickiness after | 5 | 4 | 5 |
| mixing | | | |
| Stickiness after | 5 | 3 | 7 |
| resting | | | |
| Extensibility | 5 | 5 | 5 |
| Elasticity | 5 | 6 | 4 |
| Softness | 5 | 3 | 7 |
| Baking Results | | | |
| Volume [ml] | 2262 | 2224 | 2089 |
| Standard Deviation | 11 | 40 | 15 |
| [ml] | | | |

Conclusion: The above experiment shows that use of GH12 cellulase leads to significantly improved dough properties and bread shape in comparison to GH10 type 1 and Market Rye enzyme.

### Example 7: Effect of GH12 Type 2 on Dough Properties (100% Rye Flour)

A dough was prepared according to the described baking and evaluation process using the recipe mentioned in table 6.
Dough No. 1=No enzyme
Dough No. 2=GH12 Type 2, 0.16 mg EP/kg flour
Dough No. 3=GH12 Type 2, 0.32 mg EP/kg flour

TABLE 6

| | 100% rye flour, 85% water, 2% salt, 2% yeast, | | |
|---|---|---|---|
| Recipe | 2% gluten, 1.1% citric acid (on rye flour) | | |
| Dough No. | 1 | 2 | 3 |
| Stickiness after | 5 | 4 | 4 |
| mixing | | | |
| Stickiness after | 5 | 4 | 4 |
| resting | | | |
| Extensibility | 5 | 5 | 5 |
| Elasticity | 5 | 5 | 6 |
| Softness | 5 | 4 | 4 |

Conclusion: The above experiment shows that use of GH12 cellulase leads to significantly improved dough properties.

### Example 8: Swelling Test Using Different GH Family Type Enzymes

15 grams of Rye flour (Germany) was mixed with 120 gr water and enzyme solution. The batter was divided in reagent tubes. The batters were mixed in a vortex centrifuge for 10 seconds on slow setting and 50 seconds on high setting. Then the batters were rested for 10 minutes at room temperature and centrifuged for 3 minutes in a laboratory centrifuge at 4500 rpm. The centrifuge separates the soluble and insoluble fractions, which are left to rest.

Different GH family enzymes were used, i.e. GH12 Type 1 to Type 5 (invention) as well as a GH7 cellulase, GH5 cellulase, GH11 cellulase, and four different types of GH10 cellulases. As comparative enzymes, enzymes known in the art and available as commercial products have been used.

After 18 hours, the swelling level was measured by the increase of volume of the insoluble fraction in the tube, relative to the blank. For instance, if the insoluble volume is 4 ml in the blank and 8 ml in the enzyme-treated sample, the swelling level is 1.5. The samples where there is no more clear separation of solubles and insoluble are indicated as completely swollen. The results are summarised in Table 7.

TABLE 7
Test No. 1 different GH12 enzymes

| | GH12 | GH12 | GH12 | GH12 |
|---|---|---|---|---|
| Control 1 | Type 1 | Type 5 | Type 4 | Type 3 |
| (no enzyme) | 500 ppm | 500 ppm | 500 ppm | 500 ppm |
| 1.0 | ~2.0 | 1.3 | 1.7 | 1.3 |
| | (Completely | | | |
| | swollen) | | | |

Test No. 2 different GH family enzymes

| | | | Commercial | Commercial | Commercial |
|---|---|---|---|---|---|
| | GH12 | GH12 | GH7 | GH5 | GH11 |
| Control | Type 2 | Type 1 | cellulase | cellulase | xylanase |
| (no enzyme) | 500 ppm | 500 ppm | 500 ppm | 500 ppm | 500 ppm |
| 1.0 | ~2.0 | ~2.0 | 0.6 (collapse) | 1.0 | 0.6 (collapse) |
| | (Completely | (Completely | | | |
| | swollen) | swollen) | | | |

Test No. 3 Comparison to GH10 family enzymes

| | | GH10 | GH10 | GH10 | GH10 |
|---|---|---|---|---|---|
| | GH12 | Type 1 | Type 2 | Type 3 | Type 4 |
| Control | Type 2 | 500 ppm | 500 ppm | 2000 ppm | 2000 ppm |
| (no enzyme) | 500 ppm | Thermoascus | Fusarium | Fusarium | Fusarium |
| 1.0 | ~2.0 | 0.6 | 0.6 | 0.6 | 0.9 |
| | (Completely | (collapse) | (collapse) | (collapse) | |
| | swollen) | | | | |

### Example 9: Comparative Baking Trial with GH12 Cellulase Type 2 Against No Enzyme (40% Rye Flour)

Recipe: 60% Wheat, 40% Rye, 72% water, 2% salt, 2% yeast, 1,1% citric acid (on rye flour), 2% gluten

A dough was prepared according to the described baking and evaluation process using the recipe mentioned above.
Bread No. 1=No enzyme
Bread No. 2=GH12 Type 2, 4.4 mg EP/kg flour

TABLE 8

| | Bread No. 1 | Bread No. 2 |
|---|---|---|
| Volume [ml] | 1840 | 2007 |
| Standard | 61 | 78 |
| Deviation | | |
| [ml] | | |
| Percentage | 100% | 109% |
| [%] | | |

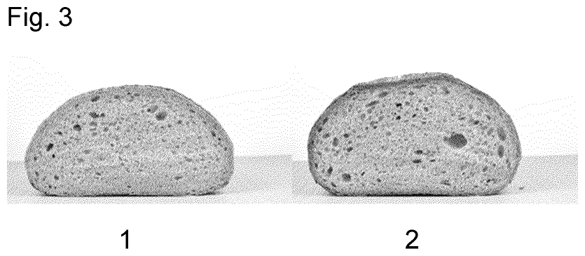

Conclusion: The above experiment shows that use of GH12 cellulase leads to significantly increased bread volume (FIG. 3).

### Example 10: Effect of GH12 Type 2 on Dough Properties (80% Rye Flour)

Recipe: 80% Rye, 20% Wheat, 76% Water, 2% Salt, 2% fresh yeast, 1,1% citric acid (on rye), 2% gluten

A dough was prepared according to the described baking and evaluation process using the recipe mentioned above.
Dough No. 1: No enzyme
Dough No. 2: GH12 Type 2, 0,008 mg EP/kg flour

TABLE 9

| | | |
|---|---|---|
| Dough No. | 1 | 2 |
| Stickiness | 5 | 3 |
| Softness | 5 | 4 |

Conclusion: The above experiment shows that use of GH12 cellulase leads to significantly improved dough properties.

1. A composition comprising an effective amount of at least one polypeptide having GH12 cellulase activity, wherein the composition improves at least one property of a dough comprising the composition, rye flour and/or of a bakery product prepared from said dough compared to a dough lacking the composition.

2. The composition of claim 1, wherein the property is selected from the stability, the stickiness, and the softness of the dough and/or, the shape, the crumb structure, and the height/width ratio of the bakery product.

3. The composition of claim 1, wherein the polypeptide having GH12 cellulase activity is derived from Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Streptomyces, Talaromyces, Thermoascus, Thielavia, Tolypocladium, Trametes, Trichoderma, Hyprocrea jecorina, Trichoderma reesei, Trichoderma viride, Trichoderma harzianum, Aspergillus niger, Aspergillus oryzae, Humicola insolens, Humicola grisea, Streptomyces sp., Streptomyces violaceoruber, Bacillus lichenformis, Bacillus amyloliquefaciens, Bacillus pumilus, Bacillus sp, Thermotoga maritima, Xanthomonas, Clostridium, Actinoplanes, Proteus, Erwinia, Shigella, Haemophilus, Streptococcus, Salmonella, Pseudomonas, Rhizobium, Agrobacterium, or Vibrio.

4. The composition of claim 1, wherein the polypeptide having GH12 cellulase activity is: GH12 Type 1 cellulase comprising SEQ ID NO:1 or NO:10, GH12 Type 2 cellulase comprising SEQ ID NO:2 or NO:3, GH12 Type 3 cellulase comprising SEQ ID NO:4 or NO:5, GH12 Type 4 cellulase comprising SEQ ID NO:6 or NO:7, GH12 Type 5 cellulase comprising SEQ ID NO:8 or NO:9 a polypeptide having GH12 cellulase activity and having at least 95% sequence identity to any one of SEQ ID NOS:1 to 10, or a mixture of at least two of said cellulases.

5. The composition of claim 1, wherein the composition further comprises an effective amount of at least one further enzyme selected from xylanase, endoxylanase, alpha amylase or maltogenic amylase.

6. The composition of claim 1, wherein the dough comprises at least 10% rye flour.

7. The composition of claim 1, wherein the polypeptide having GH12 cellulase activity is present in the dough in an amount of 0.001 to 500 mg of polypeptide per kg of flour.

8. The composition of claim 1, wherein the bakery products are crusty bakery products.

9. A method of preparing a bakery product based on rye having at least one improved property, said method comprising:
i) providing a dough that has been added an effective amount of at least one polypeptide having GH12 cellulase activity or a composition comprising at least one polypeptide having GH12 cellulase activity and rye flour, and
ii) baking the dough for a time and temperature sufficient to yield the bakery product.

10. The method of claim 9, wherein the improved property is selected from the stability, the stickiness, and/or the softness of the dough and, the shape, the crumb structure, and the height/width ratio of the bakery product.

11. The method of claim 9, wherein the polypeptide having GH12 cellulase activity is derived from Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Streptomyces, Talaromyces, Thermoascus, Thielavia, Tolypocladium, Trametes, Trichoderma, Hyprocrea jecorina, Trichoderma reesei, Trichoderma viride, Trichoderma harzianum, Aspergillus niger, Aspergillus oryzae, Humicola insolens, Humicola grisea, Streptomyces sp., Streptomyces violaceoruber, Bacillus lichenformis, Bacillus amyloliquefaciens, Bacillus pumilus, Bacillus sp, Thermotoga maritima, Xanthomonas, Clostridium, Actinoplanes, Proteus, Erwinia, Shigella, Haemophilus, Streptococcus, Salmonella, Pseudomonas, Rhizobium, Agrobacterium, or Vibrio.

12. The method of claim 9, wherein the polypeptide having GH12 cellulase activity is: GH12 Type 1 cellulase comprising SEQ ID NO:1 or NO:10, GH12 Type 2 cellulase comprising SEQ ID NO:2 or NO:3, GH12 Type 3 cellulase comprising SEQ ID NO:4 or NO:5, GH12 Type 4 cellulase comprising SEQ ID NO:6 or NO:7, GH12 Type 5 cellulase comprising SEQ ID NO:8 or NO:9, a polypeptide having GH12 cellulase activity and having at least 95% sequence identity to any one of SEQ ID NOS:1 to 10, or a mixture of at least two of said cellulases.

13. The method of claim 9, wherein the dough comprises an effective amount of at least one further enzyme selected from xylanase, endoxylanase, alpha amylase or maltogenic amylase.

14. The method of claim 9, wherein the dough comprises at least 10% rye flour.

15. The method of claim 9, wherein the polypeptide having GH12 cellulase activity has been added in an amount of 0.001 to 500 mg enzyme protein per kg of flour.

16. The method of claim 9, wherein the bakery products are crusty bakery products, preferably rye buns and loaves, whole-grain loaves, rye rolls, rye pretzels, or rye pizza.

17. A baking enzyme composition for preparing rye-based bakery products comprising a polypeptide having a GH12 cellulase activity and one or more dough or bakery product additives.

18. The baking enzyme composition of claim 17 wherein the polypeptide having GH12 cellulase activity is: GH12 Type 1 cellulase comprising SEQ ID NO:1 or NO:10, GH12 Type 2 cellulase comprising SEQ ID NO:2 or NO:3, GH12 Type 3 cellulase comprising SEQ ID NO:4 or NO:5, GH12 Type 4 cellulase comprising SEQ ID NO:6 or NO:7, GH12 Type 5 cellulase comprising SEQ ID NO:8 or NO:9, a polypeptide having GH12 cellulase activity and at least 95% sequence identity to any one of SEQ ID NOS:1 to 10, or a mixture of at least two of said cellulases.
